(19) United States
(12) Patent Application Publication
(10) Pub. No.: US 2021/0353165 A1
(43) Pub. Date: Nov. 18, 2021

(54) Pressure Assessment Using Pulse Wave Velocity

(71) Applicants: Anhui Huami Health Technology Co., Ltd., Hefei (CN); Zepp, Inc., Cupertino, CA (US)

(72) Inventors: Artem Galeev, Vancouver (CA); Yan Vule, Port Moody (CA); Yoav Aminov, Tel Aviv (IL); Kongqiao Wang, Hefei (CN)

(21) Appl. No.: 17/319,734

(22) Filed: May 13, 2021

Related U.S. Application Data
(60) Provisional application No. 63/024,811, filed on May 14, 2020.

(51) Int. Cl. A61B 5/021; A61B 5/00; G06N 20/00

(52) U.S. Cl. CPC A61B 5/02125; G06N 20/00; A61B 5/681

(57) ABSTRACT

Measuring, using a wearable device, a blood pressure of a user includes extracting, using sensor data of the wearable device, features related to a pulse wave; determining a pulse transit time (PTT); scaling at least one of the features using the PTT to obtain a scaled feature; using the scaled feature as an input to a machine-learning (ML) model; and obtaining, using an output of the ML model, the blood pressure of the user.

### CROSS REFERENCES TO RELATED APPLICATION(S)

This disclosure claims the benefit of U.S. Provisional Application No. 63/024,811, filed May 14, 2020, the disclosure of which is hereby incorporated by reference in its entirety.

### TECHNICAL FIELD

This disclosure relates generally to detecting physiological information of a user through a number of sensors of a wearable device, and more specifically to detecting blood pressure using, mainly, pressure sensors.

### BACKGROUND

Many portable devices have been developed in which sensors are used to detect variation in blood flow through arteries or blood volume in subcutaneous tissue. Applications include the monitoring of heart rate, glucose level, apnea, respiratory stress, and other physiological conditions.

High blood pressure is a major risk for heart disease. By some estimates, high blood pressure affects one in every three adults in the United States. By some other estimates, in developing and developed countries, respectively, 45% and 55% of high-blood-pressure sufferers are not aware of their condition.

The ability to monitor blood pressure via a portable (e.g., wearable) device is desirable.

### SUMMARY

Disclosed herein are implementations of a wearable device for measuring blood pressure.

A first aspect is a method for measuring, using a wearable device, a blood pressure of a user. The method includes extracting, using sensor data of the wearable device, features related to a pulse wave; determining a pulse transit time (PTT); scaling at least one of the features using the PTT to obtain a scaled feature; using the scaled feature as an input to a machine-learning (ML) model; and obtaining, using an output of the ML model, the blood pressure of the user.

A second aspect is a wearable device for measuring a blood pressure of a user. The wearable device includes a processor that is configured to extract, using first sensor data of the wearable device, a first feature related to a pulse wave in a first time window; extract, using second sensor data of the wearable device, a second feature related to the pulse wave in a second time window; determine a height difference of the wearable device, with respect to a heart of the user, between the first time window and the second time window; scale a difference between the first feature and the second feature using the height difference to obtain a scaled feature; use the scaled feature as an input to a machine-learning (ML) model; and obtain, using an output of the ML model, the blood pressure of the user.

A third aspect is a non-transitory computer-readable storage medium that includes executable instructions that, when executed by a processor, facilitate performance of operations for measuring, using a wearable device, a blood pressure of a user. The instructions include obtaining, using first sensor data of the wearable device in a first time window, a first height of the wearable device with respect to a heart of the user; obtaining, using second sensor data of the wearable device in a second time window, a second height of the wearable device with respect to the heart of the user; determining a height difference of the wearable device, with respect to a heart of the user, between the first time window and the second time window; obtaining, using an output of a machine-learning (ML) model, the blood pressure of the user; and scaling the blood pressure using the difference between the first height and the second height to obtain a scaled blood pressure of the user.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 depicts some aspects of an illustrative implementation of an apparatus according to implementations of this disclosure.

FIG. 2 depicts some aspects of an illustrative implementation of an apparatus according to implementations of this disclosure.

FIG. 3 depicts some aspects of an illustrative implementation of an apparatus according to implementations of this disclosure.

FIGS. 4A-4B depict some aspects of a user's anatomy according to implementations of this disclosure.

FIGS. 5A-5C depict some aspects of an illustrative implementation of an apparatus according to implementations of this disclosure.

FIG. 6A-6C depict some aspects of an illustrative implementation of an apparatus according to implementations of this disclosure.

FIG. 7 depicts some aspects of an illustrative implementation of an apparatus according to implementations of this disclosure.

FIG. 8 depicts an illustrative implementation of a computing system according to implementations of this disclosure.

FIG. 9 illustrates the ambiguity introduced in pulse transit time measurement when an ECG sensor is used.

FIG. 10 illustrates different configurations of wearable devices for measuring a pulse transit time (PTT) without an ECG sensor according to implementations of this disclosure.

FIG. 11 illustrates typical signals that relate to a pulse wave and from which features can be extracted according to implementations of this disclosure.

FIG. 12 is a flowchart of an example of a technique for measuring blood pressure according to an implementation of this disclosure.

FIG. 13 is a flowchart of an example of a technique for measuring blood pressure according to an implementation of this disclosure.

FIG. 14 is a flowchart of an example of a technique for measuring blood pressure according to an implementation of this disclosure.

### DETAILED DESCRIPTION

Disclosed herein are implementations of an apparatus for sensing, measuring, analyzing, and/or displaying physiological information. In one aspect, the apparatus may be a wearable device comprising an upper module and/or a lower module. The wearable device may be worn on a user's body such that one or more sensors of the upper and lower modules contact a targeted area of tissue. In one implementation, the wearable device is a watch, band, or strap that can be worn on the wrist of a user such that the upper and lower modules are each in contact with a side of the wrist.

In an embodiment, the wearable device can be a lower module that can be attached to another device. For example, the wearable device can be a lower module that is a clip and/or an add-on to a watch or another wearable device. For example, the lower module may be attachable to the bottom of a watch such that the lower module is in contact with the skin of the wearer.

Each of the upper and lower modules may comprise one or more sensors, including but not limited to optical/PPG sensors, ECG sensors/electrodes, bio impedance sensors, galvanic skin response sensors, tonometry/contact sensors, accelerometers, pressure sensors, acoustic sensors, electro-mechanical movement sensors, and/or electromagnetic sensors. In one implementation, one or more optical/PPG sensors may comprise one or more light sources for emitting light proximate a targeted area of tissue and one or more optical detectors for detecting either reflected light (where an optical detector is located on the same side of the targeted area as the light source(s), i.e., within the same module) or transmitted light (where an optical detector is located opposite the light source(s), i.e., within an opposing module).

In a further aspect, the strap or band of the wearable device may be configured so as to facilitate proper placement of one or more sensors of the upper and/or lower modules while still affording the user a degree of comfort in wearing the device. In one implementation, rather than a strap that lies in a plane perpendicular to the longitudinal axis of the user's wrist or arm (as is the case with traditional wrist watches and fitness bands), the band may be configured to traverse the user's wrist or arm at an angle that brings one or more components of the upper or lower modules into contact with a specific targeted area of the user while allowing another portion of the band to rest at a position on the user's wrist or arm that the user finds comfortable.

In another aspect, the precise location of the upper and/or lower modules can be customized such that one or more sensors of either module can be placed in an ideal location of a user, despite the physiological differences between body types from user to user.

The aforementioned features result in more comfortable wearable device while also increasing reliability and accuracy of the device sensing, measuring, analyzing, and displaying of physiological information.

In one implementation, the physiological information sensed, measured, analyzed, or displayed can include but is not limited to heart rate information, ECG waveforms, calorie expenditure, step count, speed, blood pressure, oxygen levels, pulse signal features, cardiac output, stroke volume, and respiration rate. In further implementations, the physiological information may be any information associated with a physiological parameter derived from information received by one or more sensors of the wearable device. Regardless, the physiological information may be used in the context of, for example, health and wellness monitoring, athletic training, physical rehabilitation, and patient monitoring. Of course, these examples are only illustrative of the possibilities and the device described herein may be used in any suitable context.

While the systems and devices described herein may be depicted as wrist worn devices, one skilled in the art will appreciate that the systems and methods described below can be implemented in other contexts, including the sensing, measuring, analyzing, and display of physiological data gathered from a device worn at any suitable portion of a user's body, including but not limited to, other portions of the arm, other extremities, the head, and/or the chest.

Reference will now be made in detail to certain illustrative implementations, examples of which are illustrated in the accompanying drawings. Wherever possible, the same reference numbers will be used throughout the drawings to refer to the same or like items.

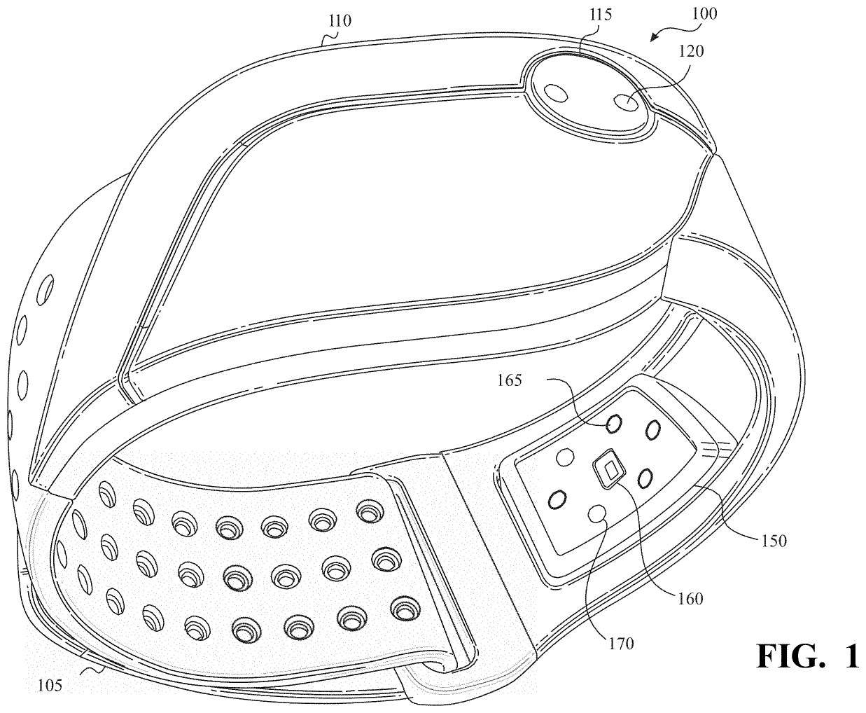

FIG. 1 depicts an illustrative implementation of an apparatus 100 according to implementations of this disclosure. In one aspect, apparatus 100 may be a physiological monitor worn by a user to sense, collect, monitor, analyze, and/or display information pertaining to one or more physiological parameters. In the depicted implementation, apparatus 100 may comprise a band, strap, or wrist watch. In further implementations, apparatus 100 may be any wearable monitor device configured for positioning at a user's wrist, arm, another extremity of the user, or some other area of the user's body.

In another aspect, apparatus 100 may comprise at least one of an upper module 110 or a lower module 150, each comprising one or more components and/or sensors for detecting, collecting, processing, and displaying one or more physiological parameters of a user and/or other information that may or may not be related to health, wellness, exercise, or physical training sessions.

In addition to upper module 110 and lower module 150, apparatus 100 may comprise a strap or band 105 extending from opposite edges of each module for securing apparatus 100 to the user. In one implementation, band(s) 105 may comprise an elastomeric material. In alternative implementations, band(s) 105 may comprise some other suitable material, including but not limited to, a fabric or metal material.

Upper module 110 or lower module 150 may also comprise a display unit (not shown) for communicating information to the user. The display unit may be an LED indicator comprising a plurality of LEDs, each a different color. The LED indicator can be configured to illuminate in different colors depending on the information being conveyed. For example, where apparatus 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees apparatus 100 through his or her peripheral vision.

In addition, or alternatively, the display unit may comprise a display screen for displaying images or characters to the user. The display unit may further comprise one or more hard or soft buttons or switches configured to accept input by the user.

Apparatus 100 may further comprise one or more communication modules. In some examples, each of upper module 110 and lower module 150 comprise a communication module such that information received at either module can be shared with the other module.

One or more communication modules can also be configured to communicate with other devices such as the user's cell phone, tablet, or computer. Communications between the upper and lower modules can be transmitted from one module to the other wirelessly (e.g., via Bluetooth, RF signal, WiFi, etc.) or through one or more electrical connections embedded in band 105. In a further implementation, any analog information collected or analyzed by either module can be translated to digital information for reducing the size of information transfers between modules. Similarly, communications between either module and another user device can be transmitted wirelessly or through a wired connection, and translated from analog to digital information to reduce the size of data transmissions.

As shown in FIG. 1, lower module 150 can comprise an array of sensors 155 including but not limited to one or more optical detectors 160, one or more light sources 165, and one or more contact pressure/tonometry sensors 170. These sensors are only illustrative of the possibilities, however, and lower module may comprise additional or alternative sensors such as one or more acoustic sensors, electromagnetic sensors, ECG electrodes, bio impedance sensors, galvanic skin response sensors, and/or accelerometers. Though not depicted in the view shown in FIG. 1, upper module 110 may also comprise one or more such sensors and components on its inside surface, i.e. the surface in contact with the user's tissue or targeted area.

In some implementations, the location of sensor array 155 or the location of one or more sensor components of sensor array 155 with respect to the user's tissue may be customized to account for differences in body type across a group of users. For example, band 105 may comprise an aperture or channel 175 within which lower module 150 is movably retained. In one implementation, lower module 150 and channel 175 can be configured to allow lower module 150 to slide along the length of channel 175 using, for example, a ridge and groove interface between the two components. In this manner, and as described in more detail below, where the user desires to place one more components of sensor array 155 at a particular location on his or her wrist, lower module 150 can be slid into the desired location along band 105. Though not depicted in FIG. 1, band 105 and upper module 110 can be similarly configured to allow for flexible or customized placement of one or more sensor components of upper module 110 with respect to the user's wrist or targeted tissue area.

In addition to the sensors and components proximate or in contact with the user's tissue, upper module 110 and/or lower module 150 may comprise additional sensors or components on their respective outer surfaces, i.e. the surfaces facing outward or away from the user's tissue. In the implementation depicted in FIG. 1, upper module 110 comprises one such outward facing sensor array 115. In one implementation, sensor array 115 may comprise one or more ECG electrodes 120. Similar to the sensor arrays of the upper and lower modules proximate or in contact with the user's tissue, outward facing sensor array 115 may further comprise one or more contact pressure/tonometry sensors, photo detectors, light sources, acoustic sensors, electromagnetic sensors, bio impedance sensors, galvanic skin response sensors, and/or accelerometers.

The outward facing sensors of sensor array 115 can be configured for activation when touched by the user (with his or her other hand) and used to collect additional information. For example, where lower module 150 comprises one or more optical detectors 160 and light sources 165 for collecting PPG and heart rate information of the user, outward facing sensor array 115 of upper module 110 may comprise ECG electrodes 120 that can be activated when the user places a fingertip in contact with the electrodes. While the optical detectors 160 and light sources 165 of lower module 150 can be used to continuously monitor blood flow of the user, outward facing sensor array 115 of upper module 110 can be used periodically or intermittently to collect potentially more accurate blood flow information which can be used to supplement or calibrate the measurements collected and analyzed by inward facing sensor array 155 of lower module 150.

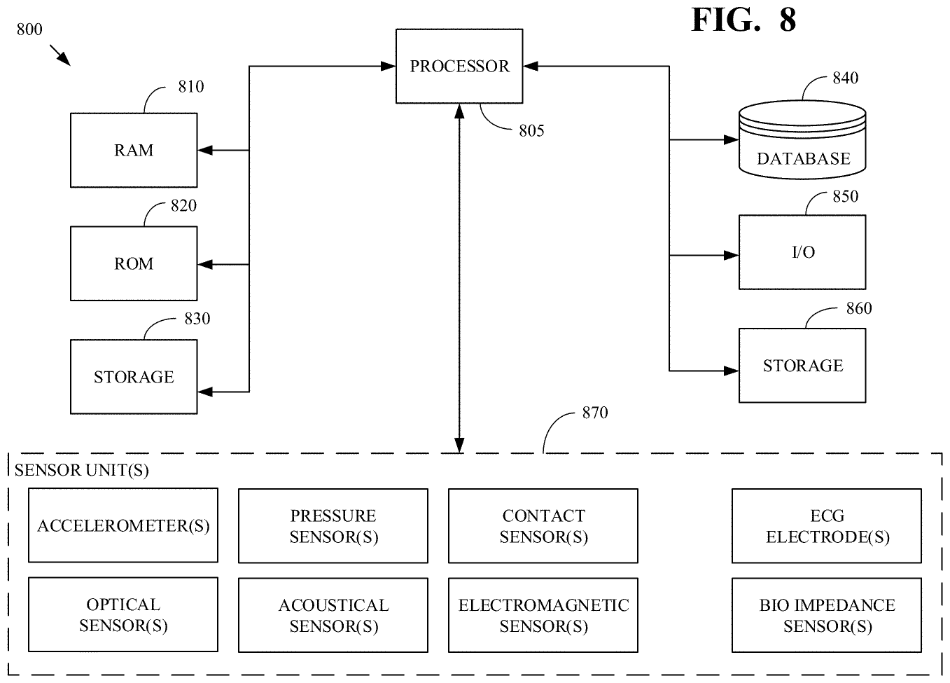

In addition to the sensor components described above with respect to each module, each module may further comprise other components for receiving, storing, analyzing, and/or transmitting physiological information. Some of those components are described below with respect to FIG. 8.

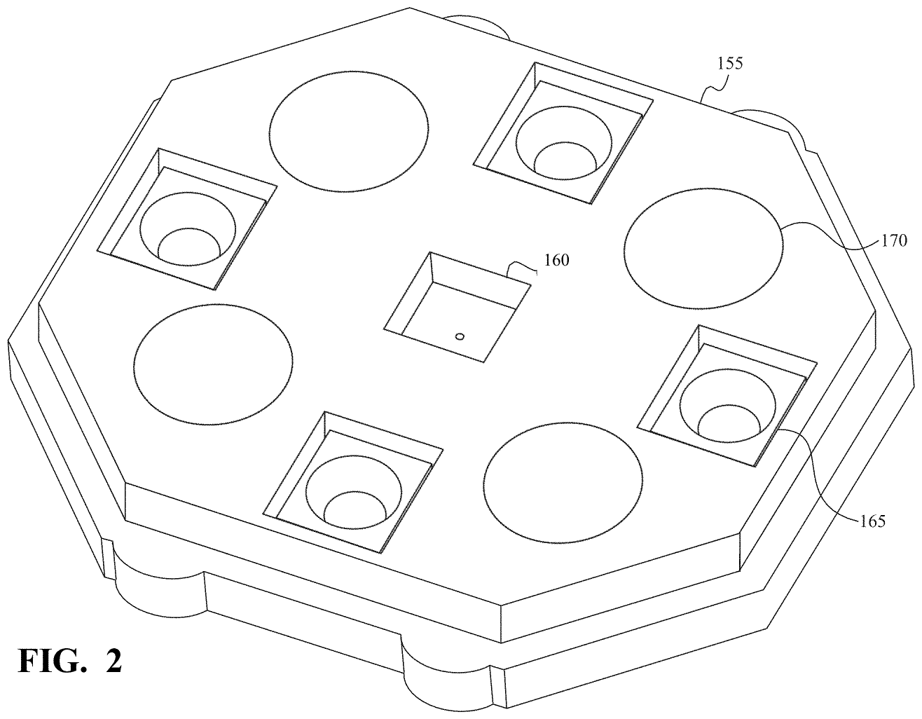

FIG. 2 depicts one implementation of inward facing sensor array 155 of lower module 150 according to implementations of this disclosure. As shown, sensor array 155 can comprise sensors including but not limited to one or more optical detectors 160, one or more light sources 165, and one or more contact pressure/tonometry sensors 170. These sensors are only illustrative of the possibilities, however, and sensor array 155 may comprise additional or alternative sensors such as one or more acoustic sensors, electromagnetic sensors, ECG electrodes, bio impedance sensors, galvanic skin response sensors, and/or accelerometers. Upper module 110 may comprise a similar inward facing sensor array (not depicted in FIG. 1) configured to position sensors proximate or in contact with the outside portion of a user's wrist or arm. In some implementations, sensor components of the upper and lower modules 110, 150 can be used in combination to collect and analyze physiological information. For example, and as described in more detail below, one or more light sources of lower module 150 can be used to transmit light through a targeted area of the user's tissue (e.g., a portion of the user's wrist) and the transmitted light can be detected by one or more photodetectors of an inward facing sensor array of upper module 110. In such an implementation, opposing modules 110 and 150 can be used to detect and analyze either reflected or transmitted light.

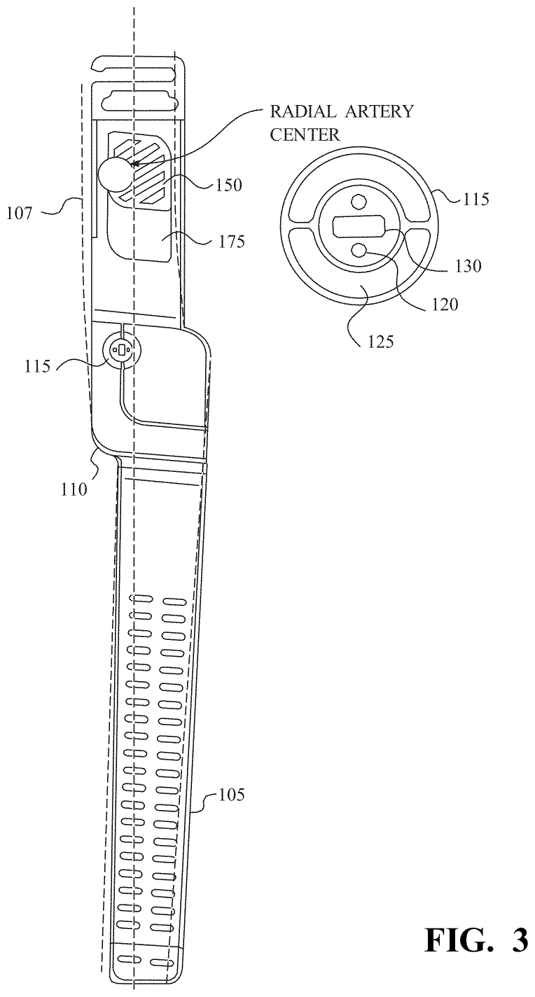

FIG. 3 depicts another view of apparatus 100 comprising band 105, upper module 110, and lower module 150 according to implementations of this disclosure. As described above, lower module 150 can be placed within channel 175 of band 105 such that lower module 150 can slide along the longitudinal axis of band 105. The movability of lower module 150 (or upper module 110 in alternative implementations) with respect to band 105 allows a user to customize the location of the inward facing sensors of lower module 150 with respect to a targeted tissue area to ensure reliable and accurate detection of physiological parameters. For example, a user can ensure that the inward facing sensors of lower module 150 are place in a location proximate the center of the user's radial artery.

In another aspect, band 105 may not extend around the user's wrist such that it traverses a circumferential path lying in a plane perpendicular to the longitudinal axis of the user's wrist or arm. Rather, the longitudinal axis of band 105 extends at an angle such that portions of inward facing sensor arrays of upper or lower modules 110, 150 can be placed at suitable locations proximate a desired targeted area of tissue while a portion of band 105 is in contact with portions of the user's wrist or arm that the user finds comfortable (i.e., above or below the wrist joint). In some implementations, where a circumferential path around a user's wrist resides in a plane perpendicular to the longitudinal extension of the user's arm or wrist, band 105 may be set at an angle 107 with respect to the perpendicular plane. In some implementations, angle 107 may be between 5° and 15° with respect to the perpendicular plane. In other implementations, angle 107 may be less than 5° or more than 15°. Of primary importance is the placement of one or more components of the sensor arrays of upper and lower modules 110, 150 proximate or in contact with a desired targeted area of tissue while allowing a portion of band 105 to be worn at a comfortable location off the user's wrist joint. Additional details regarding proper or desirable placement of one or more sensors with respect to targeted tissue areas of a user are described below with respect to other figures.

FIG. 3 also shows a closer view of outward facing sensor array 115. In the implementation depicted, sensor array 115 may comprise one or more ECG electrodes 120 for establishing an electrical connection with a user's fingertip and collected ECG data. Sensor array 115 may further comprise one or more contact pressure/tonometry sensors 125 for detecting the presence of the user's fingertip, which can trigger activation of the ECG electrodes 120. Sensor array 115 may also comprise additional or alternative components 130 such as one or more optical detectors, light sources, acoustic sensors, electromagnetic sensors, bio impedance sensors, galvanic skin response sensors, and/or accelerometers.

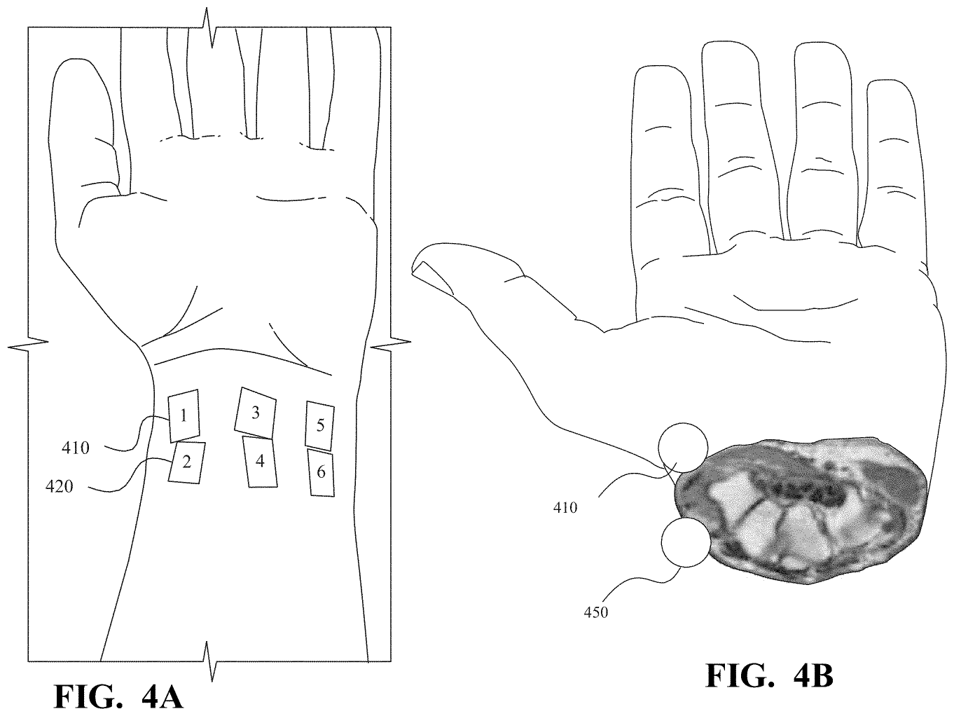

FIG. 4A depicts some points of interest on a human wrist. The best point on the wrist for detecting blood flow, for example in calculating heart rate, blood pressure, respiratory rate, etc., is at a location coinciding with the wrist joint, approximately at location 1 shown as item 410. This location is proximate the radial artery and is referred to as the CUN location.

Thus, the ideal location for a user to wear a wrist worn device is along the line across a line comprising locations 1-3-5. However, for comfort, most users prefer to wear straps or bands off the wrist joint, for example, across locations 2-4-6 shown as item 420. The result is that in most cases, users wear their monitors and corresponding sensors at a location on their wrist or arm that is not ideal and likely to introduce errors in the detection of physiological parameters.

The angle 107 of the band described with respect to FIG. 3 cures this deficiency in that it allows one or more sensor components of lower module 150 to be located above the CUN location while allowing a portion of the remaining band and/or upper module 110 to be positioned at a more comfortable location on the user's wrist or arm, such as line 2-4-6 (item 420).

Further ensuring that one or more sensors of lower module 150 can be placed at a desirable location above the CUN location, and as described in more detail above with respect to FIGS. 1 and 3, lower module 150 can slide along band 105. This allows the user to make further adjustments to the location of one or more sensors, not just along the longitudinal extension of the user's arm when apparatus 100 is in use, but also along the circumferential extension of band 105 while apparatus 100 is in use. Thus, the combination of band 105 extending around the user's wrist at an angle 107 together with the ability to slide the lower module 150 along band 105, ensure the sensors of lower module 150 can be placed at an ideal location with respect to each user (even users of different body types and physical attributes) and that the physiological parameters detected and analyzed by apparatus 100 are collected as accurately as possible.

Not only is apparatus 100 configured so as to ensure proper placement of one or more sensors and comfortability of band 105, but it also may contain additional sensors, such as a pressure sensor, at locations of apparatus 100 other than upper and lower modules 110, 150.

For example, apparatus 100 may comprise a pressure sensor located somewhere else along band 105 or at a latch that secures opposing ends of band 105 around a user's wrist for detecting pressure. Such a sensor can be used to ensure that the user is wearing the apparatus 100 tightly enough to ensure one more other sensors are in sufficient contact with a targeted area of the user's tissue to collect accurate physiological information. In alternative implementations, one or more pressure sensors of the upper and/or lower modules 110, 150 can be used to make the same determination. In either case, apparatus 100 may also be configured to alert the user (for example, via the display unit of upper module 110) if apparatus 100 is being worn too loosely or too tightly to ensure accurate measurements.

FIG. 4B depicts one example of desirable locations for one or more sensors to be placed with respect to a user's wrist or other targeted area according to implementations of this disclosure. In one implementation, one or more sensors of lower module 150 can be placed adjacent or proximate the item 410 (i.e., the CUN location) and one or more sensors of upper module 110 can be placed opposite the item 410 at point 450 of the user's wrist or targeted area. Such a configuration provides the aforementioned benefits associated with proper placement of sensors over the CUN location, but also allows for apparatus 100 to detect, collect, and analyze blood flow through the radial artery using either reflective or transmissive systems.

Wrist worn PPG sensors currently use a reflective system whereby a sensor array comprises one or more light sources and one or more optical detectors, located near one another and on the same side of a user's targeted area. The one or more light sources of the sensor array illuminate a portion of the user's tissue and light is reflected back to the optical detector(s) of the sensor array. The reflected light detected by the optical detector can be analyzed to estimate physiological parameters such as blood flow and pulse rate.

However, reflective systems may not be as accurate as transmissive systems that place one or more light sources on one side of a user's body and optical detectors on an opposing side of the user's body. One example of a transmissive system are fingertip monitors used in a clinical setting. The monitors are clipped to a patient's fingertip, one side comprising a light source for illuminating the top or bottom of the patient's fingertip, the other side comprising an optical detector for detecting the light transmitted through the fingertip.

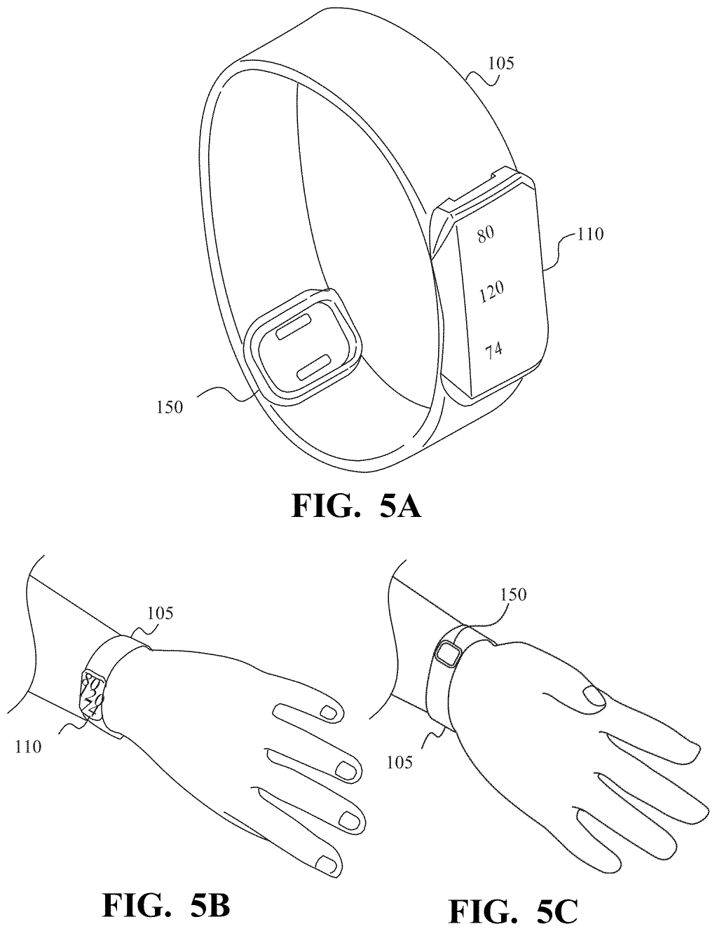

It has been thought that transmissive systems are not practical for wrist worn health monitors (or monitors worn at other locations on a user's arm or body) because the wrist is too thick for light that enters one side of a targeted area to be transmitted all the way through to the other side. However, apparatus 100 solves this problem by taking advantage of the natural location of the CUN location (the location of the radial artery at the wrist) at the inside of the wrist just under the thumb. As shown in FIGS. 5A, 5B, and 5C, apparatus 100 can be configured to place the lower module 150 comprising a light source (and/or optical detector) at the location of the CUN artery on the underside of the wrist and place the upper module 110 comprising an opposing optical detector (or light source) at a location opposite the sensors of the lower module at the periphery of the outside of the wrist just below the thumb. In this manner, the path of light transmitted through the wrist between the sensors of the lower and upper modules travels a shorter distance (shown in FIG. 4B) than if the sensors were located closer to the center of the inside and outside of a user's wrist. As a result, light illuminated from either the upper or lower module can be detected at the opposing module in a manner previously only available in clinical settings and limited to locations on the body such as the fingertip.

As described above, apparatus 100 may comprise a number of components and sensors for detecting physiological information and extracting data from it, such as blood flow, heart rate, respiratory rate, blood pressure, steps, calorie expenditure, and sleep.

Data collected from at least one or more of ECG electrodes/sensors, bio impedance sensors, galvanic skin response sensors, tonometry/contact sensors, accelerometers, pressure sensors, acoustic sensors, and electromagnetic sensors can be used for determining physiological information.

One method for determining the heart rate, respiratory rate, blood pressure, oxygen levels, and other parameters of a user involves collecting a signal indicative of blood flow pulses from a targeted area of the user's tissue. As described above, this information can be collected using, for example, a light source and a photo detector. Some implementations may use multiple light sources and they may be of varying colors (e.g., green, blue, red, etc.). For example, one light source may be an IR light source and another might be an LED light (such as a red LED). Using both an IR light source and a colored LED light (such as red) can improve accuracy as red light is visible and most effective for use on the surface of the skin while IR light is invisible yet effective for penetration into the skin. Such implementations may comprise multiple photo detectors, one or more configured to detect colored LED light (such as red) and one or more configured to detect IR light. These photo detectors (for detecting light of different wavelengths) can be combined into a single photodiode or maintained separate from one another. Further, the one or more light sources and one or more photodetectors could reside in the same module (upper or lower) in the case of a reflective system or the light source(s) could reside in one module while the optical detector(s) reside in the other in the case of a transmissive system.

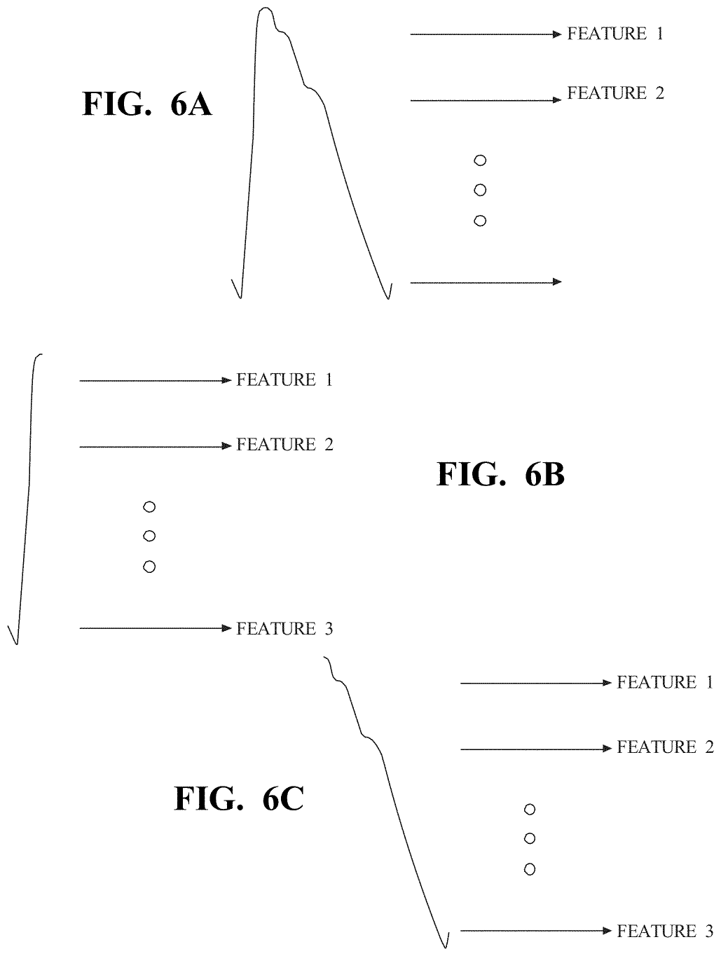

Upon collection of a blood flow pulse signal, a number of parameters can be extracted from both single pulses and a waveform comprising multiple pulses. FIG. 6A depicts a single pulse from which a number of features or parameters can be extracted. Features or parameters extracted from a single pulse can include, but are not limited to, shape of the pulse, a maximum amplitude, a minimum amplitude, a maximum derivative, a time difference between main and secondary peaks, and integral through the entire extraction time (i.e., the area under the pulse). FIGS. 6B and 6C illustrate that even portions of a single pulse can be analyzed for feature extraction. Extracting features at this level of detail has a number of advantages, including the ability to capture a great number of pulse features and store each of those features digitally without having to retain the analog waveform. The result is a savings in storage requirements and ease of data transmission.

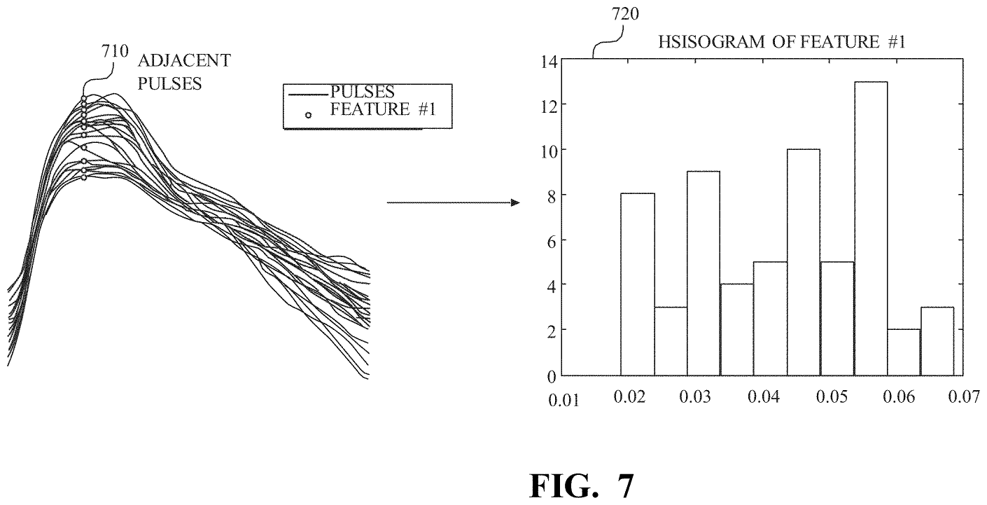

Feature extraction can also be performed on a number of pulses or a “pulse train.” FIG. 7 depicts a series of pulses overlaid with one another to show the variation among the group with respect to an identified feature according to implementations of this disclosure. In this manner, the total variation among a series of pulses with respect to a single feature can be determined. The average of a group of pulses with respect to a single feature and the standard deviation of the group with respect to the feature can also be determined. Of course, these are just examples of the types of information that can be collected from a comparison of a single feature over a group of pulses. Moreover, while FIG. 7 depicts the extraction of a single feature from the group of pulses, it should be appreciated that any number of features can be extracted from the group in a manner similar to that described above with respect to a single pulse. FIG. 7 further depicts how information collected about a single feature over a group of pulses can be digitized or presented in a histogram 720.

All of the features or parameters described above, collected using a PPG system comprising one or more light sources and/or one or more optical detectors, can be supplemented with additional sensors such as ECG electrodes/sensors, bio impedance sensors, galvanic skin response sensors, tonometry/contact sensors, accelerometers, pressure sensors, acoustic sensors, and electromagnetic sensors. For example, one or more tonometry/contact sensors can be used to extract tonometry information by measuring the contact vessel pressure. In another example, one or more acoustic sensors comprising a speaker-microphone combination (such as a micro-electro-mechanical system (“MEMS”) acoustic sensor) can be used to extract reflected sound pulses from moving vessel walls. Similarly, one or more electromagnetic sensor MEMS can be used to extract voltage induced by coils or magnet pieces pressed to moving vessel walls. In a further implementation, as described above, external or outward facing sensors can be configured to activate when touched by the off-hand (i.e., the hand on which apparatus 100 is not being worn) to collect additional information to help supplement or calibrate the information collected by the inward facing sensors of the upper or lower modules. For example, where internal facing PPG components (i.e., one or more light sources and one or more photo detectors) are used to detect reflected or transmitted light representative of blood flow pulses and some extrapolation of the data is made to determine, for example, heart rate, the user can place a fingertip of his or her off-hand on an outward facing ECG electrode (such as that shown in FIG. 1) to collect a more precise heart rate measurement. The more precise, though of more finite duration, heart rate measurement can be used to aid in the interpretation of the continuous heart rate measurements collected by the inward facing PPG sensors. The outward facing sensor can also comprise other sensors previously described herein, such as one or more contact/tonometry sensors, one or more bio impedance sensors, and one or more galvanic skin response sensors for analyzing electric pulse response. All of the information collected by an outward facing sensor from, for example, the fingertip of the user's off-hand, can be used to refine the analysis of the continuous measurements taken by any one or more of the inward facing sensors.

In addition to the inward and outward facing sensors, apparatus 100 may further comprise additional internal components such as one or more accelerometers and/or gyroscopic components for determining whether and to what extent the user is in motion (i.e., whether the user is walking, jogging, running, swimming, sitting, or sleeping). Information collected by the accelerometer(s) and/or gyroscopic components can also be used to calculate the number of steps a user has taken over a period of time. This activity information can also be used in conjunction with physiological information collected by other sensors (such as heart rate, respiration rate, blood pressure, etc.) to determine a user's caloric expenditure and other relevant information.

To determine a user's blood pressure, the PPG information described above may be combined with other sensors and techniques described herein. In one implementation, determining a user's blood pressure can comprise collecting a heart rate signal using a PPG system (i.e., one or more light sources and photo detectors) and performing feature extraction (described above) on single pulses and a series of pulses. The features extracted from single pulses and series of pulses can include statistical averages of various features across a series, information regarding the morphological shape of each pulse, the average and standard deviation of morphology of a series of pulses, temporal features such as the timing of various features within single pulses, the duration of a single pulse, as well as the average and standard deviation of the timing of a feature or duration of pulses within a series of pulses, and the timing of morphological features across a series of pulses (i.e., the frequency with which a particular pulse shape occurs in a series).

As described above, this feature extraction can not only be performed on a series of pulses and single pulses, but also on portions of a single pulse. In this manner, information pertaining to both systolic and diastolic blood pressure can be ascertained as one or more portions of an individual pulse correspond to the heart's diastole (relaxation) phase and one or more other portions of an individual pulse correspond to the heart's systole (contraction) phase. In some implementations, up to 200 features can be extracted from a partial pulse, a single pulse, and/or a series of pulses. In alternative implementations, fewer or more features may be extracted.

In addition to features extracted from PPG or ECG information, information and features can also be collected by contract/tonometry sensors, pressure sensors, bio impedance sensors galvanic skin response sensors, accelerometers, acoustic sensors, and electromagnetic sensors. For example, pressure sensors or bio impedance sensors can be used to identify blood flow pulses of user and, similar to PPG or ECG data, features can be extracted from the collected data.

The extracted features can then be cross-referenced or compared to entries in a library containing data corresponding to a population of subjects. For each subject, the library may contain information associated with each extracted feature. The library can also contain a direct measured or verified blood pressure for each subject. In further implementations, the library may contain more than one directly measured or verified blood pressure measurement for each subject, each corresponding to a subject in a different condition, such as one corresponding to the subject at rest, one corresponding to the subject engaged in light activity, and one corresponding to the subject engaged in strenuous activity. Thus, the extracted features of the user, as well as activity information pertaining to the user, can be compared to entries in the library to find one or more subjects with which the user's extracted features most closely match and the user's blood pressure can then be estimated based on the verified blood pressure of those subjects.

As one example, when features are extracted from a series of pulses, a standard deviation or range of variation across the series can be ascertained. Generally speaking, a large variation across a series of pulses can be associated with flexible, healthy veins. As a result, individuals exhibiting large pulse-to-pulse variations across a series of pulses typically have relatively low blood pressure. Conversely, little to no variation in features across a series of pulses is typically associated with relatively high blood pressure.

The library described above can be generated by extracting the same features from partial pulses, individual pulses, and series of pulses across hundreds or thousands of subjects. The subjects' verified blood pressure can also be measured such that it can be associated with each feature extracted from the subject's pulse information. The subject entries in the library can also be sorted based on information helpful for estimating blood pressure. For example, subjects in the library can be identified as male or female, belonging to a particular age group, or associated with one or more past health conditions. Individual subjects can be associated with information indicative of the subject's sex, age, weight, race, and any other medically meaningful distinction. Moreover, entries can be associated with information collected by other sensors at the time the verified blood pressure measurement was taken, including information collected by contract/tonometry sensors, pressure sensors, bio impedance sensors galvanic skin response sensors, accelerometers, acoustic sensors, and electromagnetic sensors. As just one example, if a user is determined to be engaged in physical activity (through a combination of accelerometer and heart rate data, as an example), his or her extracted features may only be compared to data in the library corresponding to subjects engaged in similar physical activity. Information pertaining to subjects contained in the library may also be correlated to each subject's resting heart rate, BMI, or some other medically significant indicia. For example, if a user is a young female with a low resting heart rate who is currently engaged in moderate activity, her extracted features should be compared to subjects in the library identified as young females with low resting heart rate whose blood pressure was verified during moderate activity rather than comparing the user's extracted features to an elderly male subject with a relatively high resting heart rate and whose blood pressure was verified during strenuous activity.

When the user's extracted features are compared to features recorded in the library, apparatus 100 can also weigh the entries of subjects most closely corresponding to the user more heavily than entries of subjects associated with indicia different from that of the user. For example, if the user is a male, features extracted from male subjects may be weighed more heavily than female subjects because a particular pulse variation in men of a particular age may correspond to relatively high blood pressure whereas the same pulse variation in women of that particular age may correspond to lower blood pressure.

According to the techniques described herein, accurate blood pressure estimates for a user can be made without requiring direct blood pressure measurement of the user. However, in some implementations, the user's blood pressure estimates can be further calibrated by direct measurement of the user's blood pressure by another device and that verified blood pressure can be input into apparatus 100 to aid in future estimations of the user's blood pressure.

Calibration can also be accomplished with an outward facing ECG sensor.

While an inward facing PPG sensor can continuously or periodically collect heart rate data of a user, occasionally the user may be prompted to place a fingertip of his or her off-hand on an outward facing ECG sensor (e.g., electrodes). The inward facing sensor arrays of apparatus 100 may contain additional electrodes thereby completing an electrical circuit through the user's body and allowing a more precise pulse waveform to be collected. Feature extraction can be performed on these pulses, series of pulses, and partial pulses in the same manner as described above with respect to PPG information and used to cross-reference the library.

In still a further implementation, where apparatus 100 determines, based on its continuous or periodic monitoring of the user's blood pressure using PPG or pressure sensors, that a user's blood pressure is unusually or dangerously high or low, apparatus 100 may prompt the user to place a fingertip of his or her off-hand on an outward facing ECG electrode in order to verify the unusual or unsafe condition. If necessary, apparatus 100 can then alert the user to call for help or seek medical assistance.

As described above, the upper and/or lower modules 110, 150 can be configured to continuously collect data from a user using its inward facing sensor arrays. However, certain techniques can be employed to reduce power consumption and conserve battery life of apparatus 100. For instance, in some implementations, only one of the upper or lower modules 110, 150 may continuously collect information. In alternative implementations, neither module may be continuously active, but may wait to collect information when conditions are such that accurate readings are most likely. For example, when one or more accelerometers or gyroscopic components of apparatus 100 indicate that a user is still, at rest, or sleeping, one or more sensors of upper module 110 and/or lower module 150 may collect information from the user while artifacts resulting from physical movement are absent.

While techniques for estimating a user's blood pressure using pulse signal, pressure, impedance, and other collected and input information has been described above, it should be appreciated that similar techniques can be employed to estimate a user's oxygen levels (SvO₂), hydration, respiration rate, and heart rate variability. For example, PPG, ECG, bio impedance, and acoustic measurements taken from the user can be cross-referenced with the aforementioned library and compared to subjects most closely matching the user (e.g., sex, age, height, weight, race, resting heart rate, BMI, current activity level, and any other medically meaningful distinction. Measured or verified hydration levels of one or more subjects can then be used to estimate the hydration level of the user. A similar process can be employed to estimate the user's oxygen levels (SvO₂), respiration rate, and heart rate variability.

FIG. 8 depicts an illustrative processor-based computing system (i.e., a system 800) representative of the type of computing system that may be present in or used in conjunction with any aspect of apparatus 100 comprising electronic circuitry according to implementations of this disclosure. Each of upper or lower modules 110, 150 may comprise any one or more components of system 800. In some implementations, one module may contain one of the components of system 800 and the other module, rather than comprising a similar component, may be in wired or wireless communication with the component residing in the other first module. Alternatively, each module may comprise a similar component as compared to the other module such that it is not necessary to communication with the first module to enjoy the functionality of the component. For example, upper module 110 may comprise storage, a power source, and/or a charging port, while lower module 150 may have access to the upper module's storage and/or draw power from the power source of the upper module through a wired or wireless connection. Alternatively, each module may have its own storage and/or power source. For the sake of simplicity, the system 800 will be described herein as if it encompasses the components of upper and lower modules 110, 150 collectively, while the reader appreciates that one or more components described herein may reside only in one module or may be found in both modules.

The system 800 may be used in conjunction with any one or more of transmitting signals to and from the one or more accelerometers, sensing or detecting signals received by one or more sensors of apparatus 100, processing received signals from one or more components or sensors of apparatus 100 or a secondary device, and storing, transmitting, or displaying information. The system 800 is illustrative only and does not exclude the possibility of another processor- or controller-based system being used in or with any of the aforementioned aspects of apparatus 100.

In one aspect, system 800 may include one or more hardware and/or software components configured to execute software programs, such as software for storing, processing, and analyzing data. For example, system 800 may include one or more hardware components such as, for example, processor 805, a random access memory module (RAM) 810, a read-only memory module (ROM) 820, a storage system 830, a database 840, one or more input/output (I/O) modules 850, an interface module 860, and one or more sensor modules 870. Alternatively and/or additionally, system 800 may include one or more software components such as, for example, a computer-readable medium including computer-executable instructions for performing methods consistent with certain disclosed implementations. It is contemplated that one or more of the hardware components listed above may be implemented using software. For example, the storage system 830 may include a software partition associated with one or more other hardware components of system 800. System 800 may include additional, fewer, and/or different components than those listed above. It is understood that the components listed above are illustrative only and not intended to be limiting or exclude suitable alternatives or additional components.

Processor 805 may include one or more processors, each configured to execute instructions and process data to perform one or more functions associated with system 800. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 8, processor 805 may be communicatively coupled to RAM 810, ROM 820, the storage system 830, database 840, I/O module 850, interface module 860, and one more of the sensor modules 870. Processor 805 may be configured to execute sequences of computer program instructions to perform various processes, which will be described in detail below. The computer program instructions may be loaded into RAM for execution by processor 805.

RAM 810 and ROM 820 may each include one or more devices for storing information associated with an operation of system 800 and/or processor 805. For example, ROM 820 may include a memory device configured to access and store information associated with system 800, including information for identifying, initializing, and monitoring the operation of one or more components and subsystems of system 800. RAM 810 may include a memory device for storing data associated with one or more operations of processor 805. For example, ROM 820 may load instructions into RAM 810 for execution by processor 805.

The storage system 830 may include any type of storage device configured to store information that processor 805 may need to perform processes consistent with the disclosed implementations.

Database 840 may include one or more software and/or hardware components that cooperate to store, organize, sort, filter, and/or arrange data used by system 800 and/or processor 805. For example, database 840 may include user profile information, historical activity and user-specific information, physiological parameter information, predetermined menu/display options, and other user preferences. Alternatively, database 840 may store additional and/or different information.

I/O module 850 may include one or more components configured to communicate information with a user associated with system 800. For example, I/O module 850 may comprise one or more buttons, switches, or touchscreens to allow a user to input parameters associated with system 800. I/O module 850 may also include a display including a graphical user interface (GUI) and/or one or more light sources for outputting information to the user. I/O module 850 may also include one or more communication channels for connecting system 800 to one or more secondary or peripheral devices such as, for example, a desktop computer, a laptop, a tablet, a smart phone, a flash drive, or a printer, to allow a user to input data to or output data from system 800.

The interface module 860 may include one or more components configured to transmit and receive data via a communication network, such as the Internet, a local area network, a workstation peer-to-peer network, a direct link network, a wireless network, or any other suitable communication channel. For example, the interface module 860 may include one or more modulators, demodulators, multiplexers, demultiplexers, network communication devices, wireless devices, antennas, modems, and any other type of device configured to enable data communication via a communication network.

System 800 may further comprise one or more sensor modules 870. In one implementation, sensor modules 870 may comprise one or more of an accelerometer module, an optical sensor module, and/or an ambient light sensor module. Of course, these sensors are only illustrative of a few possibilities and sensor modules 870 may comprise alternative or additional sensor modules suitable for use in apparatus 100. It should be noted that although one or more sensor modules are described collectively as sensor modules 870, any one or more sensors or sensor modules within apparatus 100 may operate independently of any one or more other sensors or sensor modules. Moreover, in addition to collecting, transmitting, and receiving signals or information to and from sensor modules 870 at processor 805, any one or more sensors of sensor module 870 may be configured to collect, transmit, or receive signals or information to and from other components or modules of system 800, including but not limited to database 840, I/O module 850, or the interface module 860.

Disclosed herein are also implementations of an apparatus for measuring blood pressure. Traditionally, blood pressure can be derived or calculated from a pulse transit time (PTT). PTT is typically measured using ECG technologies, or using ECG in conjunction with PPG technologies. In an example of a traditional blood pressure measurement system using PTT, PPG sensors can be used on the palm and the wrist, and ECG sensors actually sense the exact electrical pulse that is due to the pumping action of the heart. Such devices measure the time it takes the blood to travel to the Palm and to the wrist. Based on the user's physiology, the velocity of propagation can also be measured. The velocity of propagation is referred to as the pulse wave velocity (PWV). That is, the PWV measures the time that it takes a pulse released from the heart to reach, for example, the wrist. As is known, PWV is measured over a distance that is significantly longer than the length of sensing modules described herein.

However, implementations of a sensing module according to this disclosure can measure the blood pressure without the use of ECG sensors. Rather, pressure sensors can be used to measure (e.g., estimate, calculate, etc.) the blood pressure.

While the term blood pressure is used throughout, it is to be understood that blood pressure encompasses any measure relating to blood pressure such as a systolic and/or mean and/or diastolic measure. Blood pressure is related to the full shape of pulse pressure. In some cases, a maximum point (the systolic blood pressure) of a pulse cycle and/or the minimum (the diastolic blood pressure) of a pulse cycle can be determined.

As is known, the PTT and the blood pressure correlate. PTT measures the traveling time of blood from a first point in the body to a second point in the body.

Measuring the PTT accurately using wearable devices requires measuring of period/interval of blood pulse travelling time from the beginning of ejection on first measurement point toward the arrival of the pulse to the second measurement point on an artery. As mentioned above, measuring the PTT typically involves a combination of PPG and ECG sensors. The ECG impulse is the electrical signal that triggers a heart contraction.

While the ECG signal is used to approximate the aortic valve opening (AO), the ECG signal cannot and does not measure the instant of aortic valve opening. As the ECG signal measures an electrical impulse, the electrical impulse is much faster than the propagation of the blood. The electrical impulse happens before the blood ejection from the heart. The ECG impulse forces the heart to squeeze and eject the blood. However, the moment of the ECG impulse is not itself the moment that the blood ejects the blood.

The ECG signal can be almost instantaneous as compared to the blood traveling time. As such, the instant of blood ejection, as estimated by the ECG signal, includes some ambiguity period between the impulse itself and the physical ejection (i.e., AO). This ambiguity period adds distortion to the accurate PTT measurement and reduces the correlation (e.g., information, accuracy, etc.) between PTT and blood pressure and/or blood pressure changes. That is, the ambiguity as to the exact moment of the AO can reduce the accuracy of traveling time (i.e., PTT) detection.

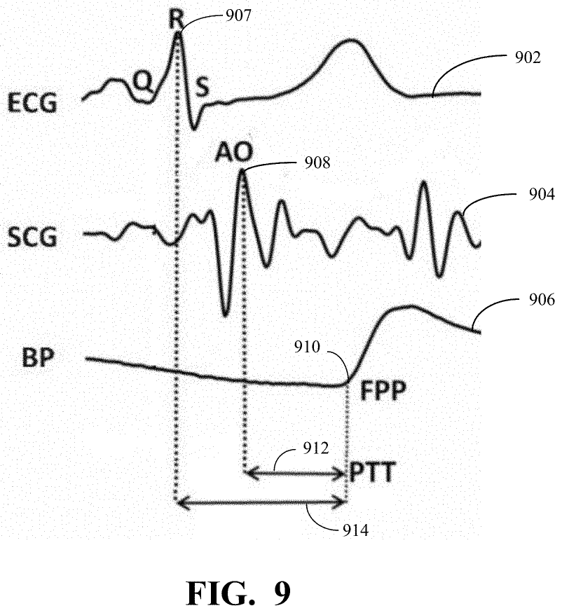

FIG. 9 illustrates the ambiguity introduced in PTT measurement when an ECG sensor is used. FIG. 9 shows a typical ECG waveform (i.e., an ECG 902), a typical SeismoCardioGram waveform (i.e., a SCG 904), and a typical blood pressure waveform (i.e., a BP 906). The SCG 904 illustrates the vibrations of the human body that are induced by the heartbeat. A peak 908 of the SCG 904 is specifically associated with the aortic valve opening. A point 910 of the BP 906 is the time of arrival of the pulse at a measurement point (e.g., a wrist, a finger, etc.). However, as mentioned above, the AO is typically approximated by the R peak in the ECG signal (i.e., an R 907 peak). A PTT 912 illustrates the true pulse transit time. However, the PTT 912 is typically estimated by a value 914, which is typically referred to as the pulse arrival time (PAT).

Wearable device configurations according to implementations described below can improve the accuracy of PTT calculation. The wearable devices described below can use one PPG sensor and another non-ECG sensor (i.e., another sensor that is not an ECG sensor) to accurately measure the PTT. The PPG sensor and the other non-ECG sensor can be placed on, or adjacent to, two different body parts that are at different distances from the heart: a first distance of the distances is relatively shorter than the second distance from the heart. That is, the first distance can be said to be short and the second distance can be said to be long. The distance between the two body parts constitutes the traveling path that is used for measuring the PTT. The PPG sensor and the other non-ECG sensor can be used in a non-invasive and comfortable way. That is, the wearable device configurations allow for comfortable blood pressure spot (i.e., non-continuous) measurement of a user without having the user change his or her wearing habits of the wearable device.

In a configuration, the wearable device can be connected (e.g., wirelessly connected) to another device that includes a PPG sensor and from which the wearable device can receive a PPG sensor measurement so that the wearable device can measure the blood pressure or a change in blood pressure.

The wearable device configurations described below can eliminate the ejection ambiguity by not using an ECG sensor. By using similar sensors at a first body location and a second body location to measure the pulse traveling time between the first body location and the second body location, the PTT measurement can be more accurate than using a PPG and an ECG sensor. The difference in arrival time of a pulse at the first and second body locations and the distance between the first and the second locations can be used to calculate the PTT. The distance between the first and the second locations can be obtained using (e.g., can be based on, etc.) a personal profile of the user wearing the wearable device. Thus, the length of the artery between the first and the second locations can be estimated.

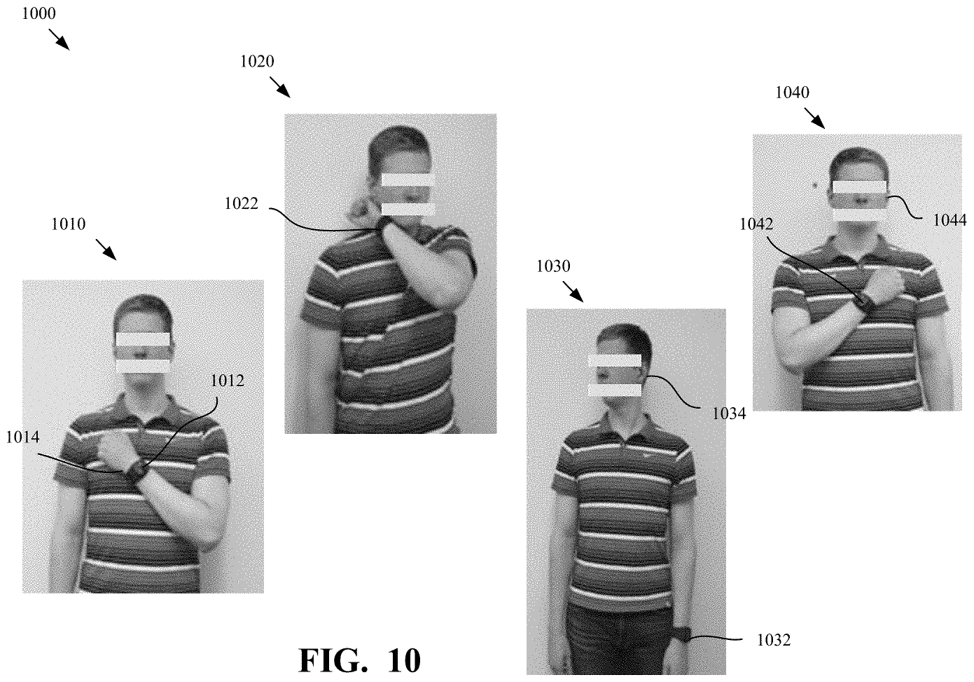

FIG. 10 illustrates different configurations 1000 of wearable devices for measuring a pulse transit time (PTT) without an ECG sensor according to implementations of this disclosure. Wearable devices as described with respect to FIG. 10 reduce the uncertainty (e.g., ambiguity) of measuring the PTT associated with using a PPG sensor and an ECG sensor by measuring (such as by using an ultrasound or physical motion sensor) the ejection itself. The configurations described below present user interface (UI) concepts that allow a user to measure the user's blood pressure without additional devices. In some situations, minor arm manipulation may be required.

In a configuration 1010, a user is shown as wearing a wearable device 1012, which can be as described above. The wearable device 1012 is worn on a wrist 1014 of the user. The wearable device 1012 includes a lower module that includes a first sensor that is a PPG sensor and a second sensor for detecting blood ejection from the heart. The PPG sensor can be placed above the CUN location, as described above.

The second sensor for detecting blood ejection from the heart can be a sensor for detecting a physical motion of the chest at the time of the moment of the aortic valve opening. In an example, the second sensor can be an accelerometer.

Thus when the user places the user's wrist on the chest during spot (e.g., episodical) measurement of the blood pressure, the chest can be the first measurement point and the wrist can be the second measuring point. That is, at the first measurement point, the heart contraction can be measured (e.g., by the accelerometer); and at the second measurement point, the arrival time at the wrist can be measured using the PPG sensor.

In a configuration 1020, a user is shown as wearing a wearable device 1022, which can be as described above. The wearable device 1022 is worn on a wrist of the user. The wearable device 1022 includes a lower module (not specifically shown) that includes a first PPG sensor and an upper module that includes a second PPG sensor. The first PPG sensor can be placed above the CUN location, as described above. The second PPG sensor can be configured for placement over the carotid artery. In a configuration, the wearable device 1022 can be a watch and the second PPG sensor can be embedded in the face of the watch.

Thus, in the configuration 1020, the first measurement point can be the carotid artery (e.g., at the neck) and the second measurement point can be the wrist (e.g., the CUN location) of the user. Thus, a pulse that travels from the heart can first be detected (e.g., arrival time) at the first measurement point at a first time and then detected at the second measurement point at a second time. As a first distance from the heart to the neck and a second distance from the heart to the wrist are known, based on a personal profile of the user wearing the wearable device, the distance between the first measurement point (e.g., the neck) and the second measurement point (e.g., the wrist) can be calculated as the difference between the first distance and the second distance. Thus, the PTT can be calculated using the distance and the respective arrival times at the first measurement location and the second location.

In a configuration 1030, a user is shown as wearing a wearable device 1032, which can be as described above. The wearable device 1032 is worn on a wrist of the user. The wearable device also includes (e.g., is in communication with) an ear device 1034 (e.g., a device that can be inserted in the ear). The ear device includes the first PPG sensor. The ear device can be an earbud (a tiny speaker that can be worn inside an ear), a headphone, a hearing aid, or some other ear device or ear attachment. The wearable device 1032 includes a lower module (not specifically shown) that includes a second PPG. The second PPG sensor can be placed above the CUN location, as described above.

Thus, the first measurement location is at the ear and the second measurement location is at the wrist. The first measurement location is at a first distance from the heart, which is shorter than a second distance of the second measurement location from the heart. In such configuration, the wearable device can perform continuous measurement of the blood pressure. The user would not be required to perform any special movement for the continuous measurement to be performed. The continuous measurement of the blood pressure can be performed so long as the ear device 1034 is inserted in or attached to the user's ear.

In another configuration, instead of an ear device, the first PPG sensor can be part of a necklace that can be worn around the neck of the user. The necklace can be configured such that the first PPG sensor is placed over the carotid artery. In such a configuration, continuous measurement of the blood pressure can be performed. In yet another configuration, the first PPG sensor can be part of another wrist-worn device. Thus, the wearable device can be worn on one wrist (e.g., the left or right wrist) and the wrist-worn device can be worn on the other wrist (e.g., the right or left wrist).

In a configuration 1040, a user is shown as wearing a wearable device 1042, which can be as described above. The wearable device 1042 is worn on a wrist of the user. The wearable device 1042 includes a lower module that includes an accelerometer or an ultrasound sensor, as described with respect to the configuration 1010. The wearable device 1042 also includes (e.g., is in communication with) an ear device 1044, which can be as described with respect to the ear device 1034. The ear device includes a second PPG sensor. Thus, the first measuring location can be at the heart to detect the aortic opening and the second measuring location can be the ear.

Other configurations of the wearable device are also possible. For example, in an example of the configuration 1010, a third measurement point can be in an ear with second PPG sensor. Information from the two PPG sensors can be used to augment each other. For example, the measurements from the two PPG sensors can be averaged. In another configuration, a second device (e.g., attachable device, wearable device, etc.) that includes a second PPG sensor can be attached to a body part of the user's choosing.

Features of the pulse wave of a user can be used to predict (e.g., estimate, etc.) the blood pressure of a user. For example, features extracted from the pulse wave can be used as inputs to a machine-learning (ML) model that is trained to output a blood pressure estimate from the input features. In some examples, differences in features, such as the differences between features extracted at two different time points or in two different time windows, can be used as inputs to a ML model to output a change in the blood pressure between the two time points. In addition to the pulse wave features, personal features (e.g., age, gender, body type, etc.) to the user can also be used to input to the ML models.

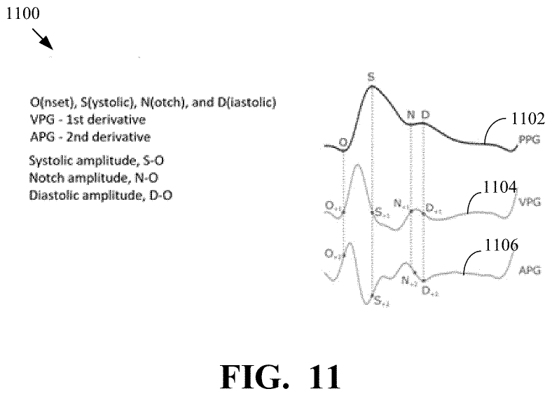

FIG. 11 illustrates typical signals 1100 that relate to a pulse wave and from which features can be extracted according to implementations of this disclosure. Extracted features can be absolute values of points of interest of one or more of the signals 1100, ratios of values related to different points of interest, differences between the features of one wave, other features, or a combination thereof.

A PPG waveform 1102 illustrates the onset O that is the start of the systolic phase and the peak (S) that is the end of the systolic phase. The diastolic notch is indicated by the (N). The pulse wave diastolic peak is marked by (D). The pulse wave end (PWE) (not specifically shown) is indicated by a valley at the end of the diastolic phase. A local maximum or an inflection point between the peak and the PWE marks the pulse wave diastolic peak (PWDP). The vertical amplitude distance between the onset (O) and peak (S), (i.e., S-O) is the systolic amplitude. The difference between diastolic notch (N) and the onset (O) (i.e., N-O) is the notch amplitude. The difference between the diastolic peak (D) and the onset (O) (i.e., D-O) is the diastolic amplitude. The horizontal distance between the onset (O) and the PWE is the pulse wave duration (PWD). Each of these can be used as features to be used as input to the ML model.

Features can also be extracted from the velocity of PPG (VPG) signal 1104 and/or the Acceleration PlethysmoGram (APG) signal 1106. The VPG signal 1104 is the first derivative of the PPG and represents the velocity of blood at a point of measurement of the pulse wave (e.g., wrist, finger, etc.). The APG signal 1106 is the second derivative of the PPG signal and represents the acceleration of blood flow at the point of measurement of the pulse wave.

The PTT, as measured using device configurations as described with respect to FIG. 10, can be used as a scaling factor. The PTT can be used to scale features extracted from the signals that relate to a pulse wave, such as the signals 1100 described with respect to FIG. 11.

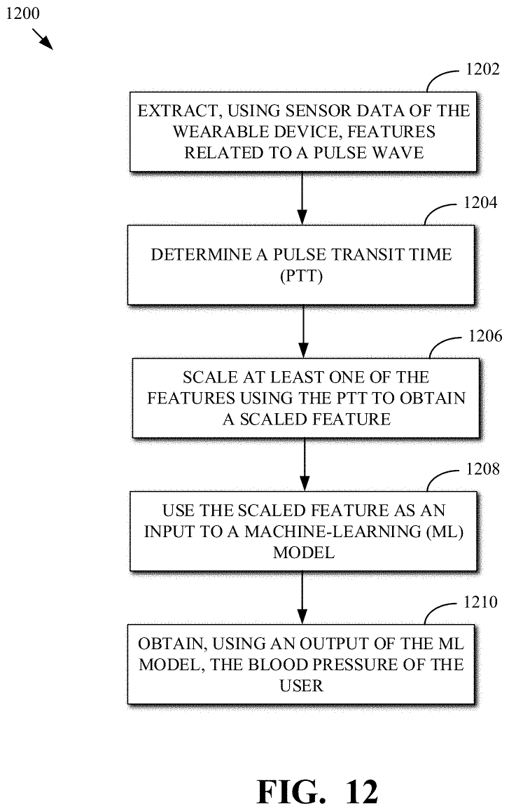

FIG. 12 is a flowchart of an example of a technique 1200 for measuring blood pressure according to an implementation of this disclosure. The technique 1200 can be implemented by a wearable device that is worn by a user, such as the apparatus 100 of FIG. 1 or the apparatus 900 of FIG. 9. The technique 1200 can be implemented as executable instructions that can be stored in a memory, such as one of the storage system 830 or the ROM 820 of FIG. 8. The instructions can be executed by a processor, such as the processor 805 of FIG. 8, to perform the steps of the technique 1200. The technique 1200 can be implemented using specialized hardware or firmware.

At 1202, the technique 1200 extracts, using sensor data of the wearable device, features related to a pulse wave. The features can be one or more of the features described with respect to FIG. 11. The features can include one or more of an amount of pressure that blood exerts on a skin surface of the user, a pulse front velocity, a pulse onset time, more features, fewer features, other features, or a combination thereof. For example, the wearable device can include sensors for measuring blood flow, the pressure that the blood exerts on the veins and on the skin surface of the user, velocity of the blood flow, amplitude of the pulse of the user, amplitude of PPG signals, and so on. In an example, the features can also include personal features to the user, such as age, gender, body type, weight, height, more, fewer, other personal features, or a combination thereof. Such personal features may not be extracted using sensors of the wearable device.

In an example, the features in a time window can be features extracted based on one sampling of the sensors. In another example, several samples of sensor data can be obtained in the time window and the features can be extracted from the several samples. For examples, the several samples can be averaged. As used in this disclosure, the term time point also encompasses the term time window since any number measurements related to one feature taken in a time window can be collapsed to (e.g., combined into, etc.) one measurement similar to one measurement taken at one time point.

In an example, the technique 1200 can estimate a change in blood pressure. As such, the features related to a pulse wave can be feature differences between two time points (or, equivalently, two time windows). Features differences are calculated for at least some of the features extracted from sensor data. However, personal features can be used as is. To illustrate, assume that only two features (F₁ and F₂) are extracted from the sensor data to be used for estimating the blood pressure change. Thus, the first features at the first time window can be F₁^{t=1} and F₂^{t=1}; the second features at the second time window can be F₁^{t=2} and F₁^{t=2}; and the features differences can be (F₁^{t=2}−F₁^{t=1}) and (F₂^{t=2}−F₂^{t=1}). These features differences are used as inputs to the ML model. As can be appreciated, many predictive features are possible. The term “feature differences,” as used herein, is not limited to subtraction of the respective feature values. Rather “feature differences” encompasses other ways of combining the features, other than subtraction. For example, ratios (e.g., F₁⁻²/F₁^{t=2} and F₁^{t=2}/F₁^{t=1}) of the respective feature values, logarithmic functions of the respective features, or other functions (or feature manipulations) of the respective features can be used.

At 1204, the technique 1200 determines a first pulse transit time (PTT). Determining the PTT can include obtaining, using a first sensor of the wearable device, a first pulse arrival time at a first body location of the user; obtaining, using a first PhotoPlethysmoGraphic (PPG) sensor of the wearable device, a second pulse arrival time at a second body location of the user; and obtaining the PTT using the first pulse arrival time and second pulse arrival time.

In an example, the first sensor can be a second PPG sensor and the first body location is an ear of the user, and the first PPG sensor can be an optical sensor that is placed over and facing a radial artery of the user. In an example, the first PPG sensor can be an optical sensor and the first body location is an ear of the user, and the first sensor can be placed over a heart of the user to detect a time of contraction of the heart. In an example, the first sensor can be a second PPG sensor of the wearable device, and the first body location of the user can be a carotid artery of the user. In an example, the first sensor of the wearable device can be placed over a heart of the user to detect a time of contraction of the heart, and the first PPG sensor can be an optical sensor that is placed over and facing a radial artery of the user. The first sensor for detecting a time of contraction of the heart can be an accelerometer or a pressure sensing device.

At 1206, the technique 1200 scales at least one of the features using the PTT to obtain a scaled feature. As mentioned above, the PTT is the interval between the ejection and the arrival at a PPG sensor. Short term variability of the PTT can depend mainly on the force of the heart contraction. Thus, the PTT can depend on the stroke volume variability. As such, the PTT can be used as a scaling factor of at least some of the extracted features. In an example, the absolute value of the PTT can be used as the scaling factor. In another example, the variability (e.g., change from one sampling time or window to another) can be used as the scaling factor. Scaling the features (or feature differences) using the PTT (or change in PTT) can have the effect of adding into, or removing from, the extracted features the contribution of the stroke volume.

To illustrate, assume that the feature F₁ denotes the local force from an artery to a sensor located over the artery. Thus, F₁ is a function of the blood pressure (BP) and the PTT (i.e., F₁≈f(BP, PTT)). Correspondingly, the blood pressure BP is a function of the PTT and the feature, F₁ (i.e., BP≈g(PTT, F₁)). Using the local force feature F₁ is a mere example and any other feature (e.g., property of the pulse wave) can be used. As mentioned above, in the case that the change in blood pressure is being estimated, the change in PTT and the change in the features can be used. In an example, the first derivatives of the PTT and the features can be used. Thus, for example, BP′=g(PTT′, F₁′). In another example, the second derivatives of the PTT and the features can be used.

Scaling can mean multiplying the feature by the PTT. Scaling can mean dividing the feature by the PTT. Scaling can mean multiplying the feature difference by the PTT difference. Scaling can mean dividing the feature difference by the PTT difference. Thus, for example, given a feature F₁ with two measurements (F₁¹ and F₁²) and two measurements of the PTT (PTV and PTT²), then the scaled feature can be calculated using equation (1).

$$Scaled Feature = \frac{{F}_{1}^{1}}{{F}_{1}^{2}} \times \frac{P T {T}^{1}}{P T {T}^{2}} \qquad ( 1 )$$

In an example, if two measurements of a features are obtained and a blood pressure value (BP) was obtained at the first measurement, then the scaled feature can be obtained using equation (2), where Coef can be used as a temporary variable.

$$\{ \begin{matrix}C o e f = \frac{{F}_{1}^{1} \times B P}{P T {T}^{1}} \\ Scaled Feature = \frac{P T {T}^{2} \times C o e f}{{F}_{1}^{2}}\end{matrix} \qquad ( 2 )$$

To reiterate, the purpose of the scaling is compensate a change of feature related to change of PTT or to scale a feature to some range related to PTT and blood pressure. Also, to reiterate, the features to be scaled can be absolute values of points of interest of the waveforms related to the pulse wave, differences between points of interest, ratios of values related to different points of interest, other features, or a combination thereof.

At 1208, the technique 1200 uses the scaled feature as an input to a machine-learning (ML) model. The ML model can be trained to output an estimate (e.g., a prediction) of a blood pressure based on the inputs, including the scaled feature. In an example, the ML model can be trained to output an estimate of a change in blood pressure between two time points. Thus, the scaled feature can be a scaled difference of two measurements of the feature, obtained at two time points (or time windows), where the feature is scaled using a difference between two measurements of the PTT corresponding to the time points (or time windows).

At 1210, the technique 1200 obtains, using an output of the ML model, the blood pressure of the user. In an example, a change in blood pressure is obtained from the ML model. The change in blood pressure can be added to a baseline blood pressure. The baseline blood pressure value can be provided to the wearable device. Thus, the technique 1200 can further include determining another PTT at a second time point and obtaining a second scaled feature at the second time point. Using the scaled feature as an input to the ML model can include scaling a first difference of the PTT and the another PTT and a second difference of the scaled feature and the second scaled feature.

For example, during a calibration window (e.g., an initial time window) of the wearable device, the user may be asked to provide a baseline blood pressure value to the wearable device or to a device that is in communication with the wearable device. The baseline blood pressure may be obtained by the user using traditional blood pressure measurement techniques and instruments (e.g., an ECG device, a sphygmomanometer, other Korotkov-sounds-based device or technique). The calibration can have, or can be at, different frequencies. In an example, the calibration window can be a one-time calibration window, such as at an initial setup time of a wearable device after the user purchases the wearable device. In an example, the calibration window can have a recurring frequency (e.g., daily, such as every morning, weekly, or some other frequency). In an example, the calibration can be explicitly initiated by the user at any time.

In some implementations, the acceleration and/or deacceleration of blood due to gravitation can be considered. It is noted that if a point (e.g., location) of a PPG sensor is higher than the heart level, the PTT can be longer (in time) due to acceleration of blood. Correspondently, if a point of a PPG sensor is lower than the heart level, then the PTT can be shorter (in time).

A factor related to the acceleration and/or deceleration can be used as a scaler (e.g., a second scaling factor) of the extracted features. For example, a first measurement of a feature may be measured at a wrist (e.g., feature value at a first level, denoted FL₁) when the wrist was placed at the heart level (e.g., Level₁) of the user. A second measurement at the wrist (e.g., the value of the feature value at a second level, denoted FL₂) may be measured while the wrist is placed at a table (Level₂), which is below the heart level. The feature can be scaled similarly to as described above with respect to scaling using the PTT using (Level₁-Level₂, in cm) as a height scaling factor. In an example, a feature can be scaled using equation (3).

$$Scaled Feature = F {L}_{2} - \frac{F {L}_{2} - F {L}_{1}}{C o e f \times ( Level 1 - Level 2 )} \qquad ( 3 )$$

In equation (3), Coef indicates the change of local blood pressure due to elevating or lowering of the wearable device (e.g., the wrist if the wearable device is worn on the wrist). The coefficient Coef is derived from the hydrostatics of the blood. For example, usually Coef=0.7 is used indicating an increase of local blood pressure of +7 mmHg in the wrist with the lowering by −10 cm of the wrist below the heart level.

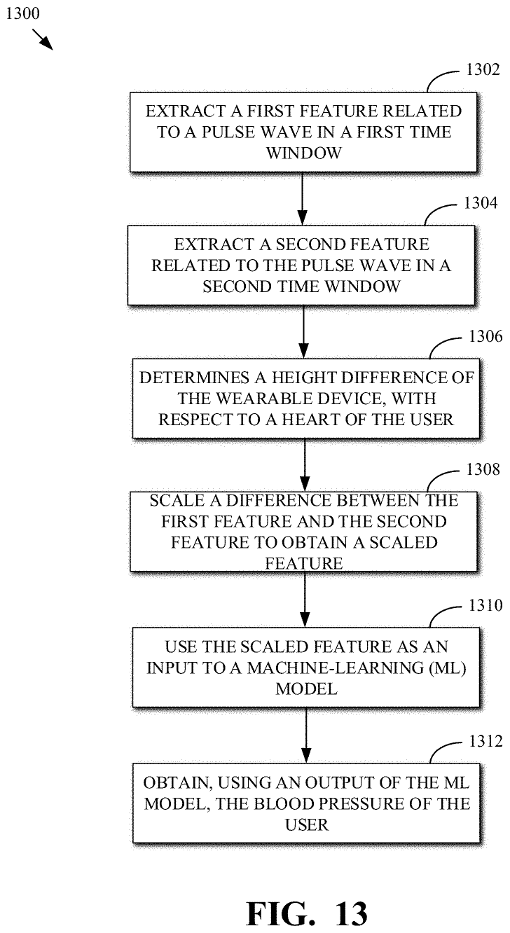

FIG. 13 is a flowchart of an example of a technique for measuring blood pressure according to an implementation of this disclosure. The technique 1300 can be implemented by a wearable device that is worn by a user, such as the apparatus 100 of FIG. 1 or the apparatus 900 of FIG. 9. The technique 1300 can be implemented as executable instructions that can be stored in a memory, such as one of the storage system 830 or the ROM 820 of FIG. 8. The instructions can be executed by a processor, such as the processor 805 of FIG. 8, to perform the steps of the technique 1300. The technique 1300 can be implemented using specialized hardware or firmware.

At 1302, the technique 1300 extracts, using first sensor data of the wearable device, a first feature related to a pulse wave in a first time window. At 1304, the technique 1300 extracts, using second sensor data of the wearable device, a second feature related to the pulse wave in a second time window.

At 1306, the technique 1300 determines a height difference of the wearable device, with respect to a heart of the user, between the first time window and the second time window. At 1308, the technique 1300 scales a difference between the first feature and the second feature using the height difference to obtain a scaled feature. The difference can be scaled using a coefficient that indicates a change of local blood pressure due to elevating or lowering of the wearable device. At 1310, the technique 1300 uses the scaled feature as an input to a machine-learning (ML) model. At 1312, the technique 1300 obtains, using an output of the ML model, the blood pressure of the user.

In an example, the wearable device can include one or more sensors for measuring the location of the wearable device (and therefore the location of the first PPG sensor) as compared to the heart of the user. The additional sensor can be a barometer. In another example, the difference can be provided to (e.g., entered into) the wearable device by the user.

In another example, the location of the first PPG sensor can be determined using image recognition. For example, an image of the user (e.g., a self-image or selfie) can be captured, such as using a camera of a device of the user. The image can be used to measure a distance between the heart of the user and the first PPG sensor (e.g., the wrist) using image and/or video processing.

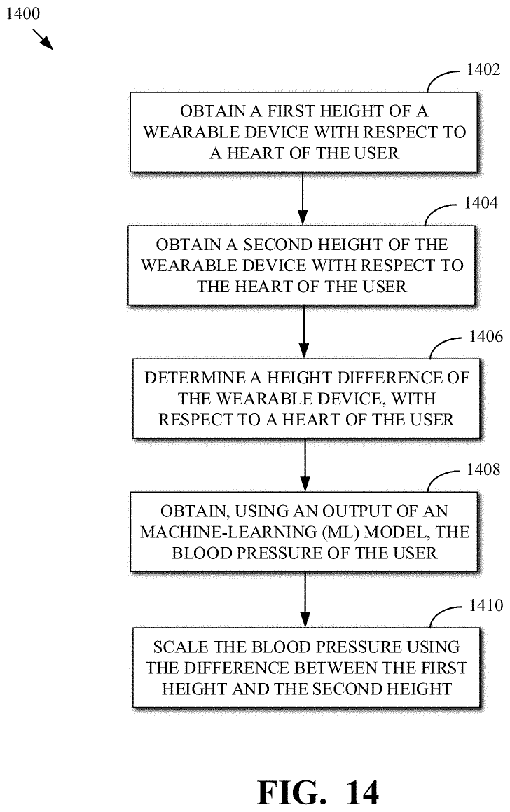

FIG. 14 is a flowchart of an example of a technique 1400 for measuring blood pressure according to an implementation of this disclosure. In this aspect of the disclosed implementations, a blood pressure obtained using the machine model can be scaled based on the difference between the first height and the second height to obtain a scaled blood pressure of the user. That is, instead of scaling the feature differences based on the height difference and using the scaled features as input to the ML model, the blood pressure that is obtained from the ML model using the feature differences can itself be scaled based the height difference.

The technique 1400 can be implemented by a wearable device that is worn by a user, such as the apparatus 100 of FIG. 1 or the apparatus 900 of FIG. 9. The technique 1400 can be implemented as executable instructions that can be stored in a memory, such as one of the storage system 830 or the ROM 820 of FIG. 8. The instructions can be executed by a processor, such as the processor 805 of FIG. 8, to perform the steps of the technique 1400. The technique 1400 can be implemented using specialized hardware or firmware.

At 1402, the technique 1400 obtains, using first sensor data of the wearable device in a first time window, a first height of the wearable device with respect to a heart of the user, as described above. At 1404, the technique 1400 obtains, using second sensor data of the wearable device in a second time window, a second height of the wearable device with respect to the heart of the user, as described above.

At 1406, the technique 1400 determines a height difference of the wearable device, with respect to a heart of the user, between the first time window and the second time window, as described above. At 1408, the technique 1400 obtains, using an output of a machine-learning (ML) model, the blood pressure of the user, as described above. At 1410, the technique 1400 scales the blood pressure using the difference between the first height and the second height to obtain a scaled blood pressure of the user. The blood pressure can be scaled using a coefficient that indicates a change of local blood pressure due to elevating or lowering of the wearable device. The height difference can be determined using image processing, as described above. The height difference can be determined using sensor data (e.g., sensor data from a barometer) in the first window and the second window, as described above.

In an example, the height difference of the wearable device can be used as another input to the ML model.

While implementations have been illustrated and described, it will be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for measuring, using a wearable device, a blood pressure of a user, comprising:
extracting, using sensor data of the wearable device, features related to a pulse wave;
determining a pulse transit time (PTT);
scaling at least one of the features using the PTT to obtain a scaled feature;
using the scaled feature as an input to a machine-learning (ML) model; and
obtaining, using an output of the ML model, the blood pressure of the user.

2. The method of claim 1, wherein determining the pulse transit time comprises:
obtaining, using a first sensor of the wearable device, a first pulse arrival time at a first body location of the user;
obtaining, using a first photoplethysmographic (PPG) sensor of the wearable device, a second pulse arrival time at a second body location of the user; and
obtaining the PTT using the first pulse arrival time and second pulse arrival time.

3. The method of claim 2,
wherein the first sensor is a second PPG sensor and the first body location is an ear of the user, and
wherein the first PPG sensor is an optical sensor that is placed over and facing a radial artery of the user.

4. The method of claim 2,
wherein the first PPG sensor is an optical sensor and the first body location is an ear of the user, and
wherein the first sensor is placed over a heart of the user to detect a time of contraction of the heart.

5. The method of claim 2,
wherein the first sensor of the wearable device is placed over a heart of the user to detect a time of contraction of the heart, and
wherein the first PPG sensor is an optical sensor that is placed over and facing a radial artery of the user.

6. The method of claim 5, wherein the first sensor is at least one of an accelerometer or a pressure sensing device.

7. The method of claim 2, wherein the first sensor is a second PPG sensor of the wearable device, and wherein the first body location of the user is a carotid artery of the user.

8. The method of claim 1, wherein scaling the at least one of the features using the PTT to obtain the scaled feature comprises:
performing one of multiplying or dividing the scaled feature and the PTT.

9. The method of claim 1,
wherein the output of the ML model is a change in blood pressure,
wherein the PTT is determined at a first time point,
the method further comprising:
determining another PTT at a second time point; and
obtaining a second scaled feature at the second time point,
wherein using the scaled feature as an input to a machine-learning (ML) model comprises:
scaling a first difference of the PTT and the another PTT and a second difference of the scaled feature and the second scaled feature.

10. A wearable device for measuring a blood pressure of a user, comprising:
a processor configured to:
extract, using first sensor data of the wearable device, a first feature related to a pulse wave in a first time window;
extract, using second sensor data of the wearable device, a second feature related to the pulse wave in a second time window;
determine a height difference of the wearable device, with respect to a heart of the user, between the first time window and the second time window;
scale a difference between the first feature and the second feature using the height difference to obtain a scaled feature;
use the scaled feature as an input to a machine-learning (ML) model; and
obtain, using an output of the ML model, the blood pressure of the user.

11. The wearable device of claim 10, wherein to determine the height difference of the wearable device, with respect to the heart of the user, between the first time window and the second time window comprises to:
obtain, using a camera in the first time window, a first image showing a first height of the wearable device with respect to the heart of the user;
obtain, using the camera in the second time window, a second image showing a second height of the wearable device with respect to the heart of the user; and
determine the height difference of the wearable device, with respect to the heart of the user, between the first height and the second height using digital image processing.

12. The wearable device of claim 10, wherein to determine the height difference of the wearable device, with respect to the heart of the user, between the first time window and the second time window comprises to:
obtain, using a sensor of the wearable device in the first time window, a first height of the wearable device with respect to a heart of the user;
obtain, using the sensor of the wearable device in a second time window, a second height of the wearable device with respect to the heart of the user; and
determine the height difference of the wearable device, with respect to a heart of the user, between the first height and the second height.

13. The wearable device of claim 12, wherein the sensor is a barometer.

14. The wearable device of claim 10, wherein the difference is scaled using a coefficient that indicates a change of local blood pressure due to elevating or lowering of the wearable device.

15. A non-transitory computer-readable storage medium, comprising executable instructions that, when executed by a processor, facilitate performance of operations for measuring, using a wearable device, a blood pressure of a user, the instructions comprising:
obtaining, using first sensor data of the wearable device in a first time window, a first height of the wearable device with respect to a heart of the user;
obtaining, using second sensor data of the wearable device in a second time window, a second height of the wearable device with respect to the heart of the user;
determining a height difference of the wearable device, with respect to a heart of the user, between the first time window and the second time window;
obtaining, using an output of a machine-learning (ML) model, the blood pressure of the user; and
scaling the blood pressure using the difference between the first height and the second height to obtain a scaled blood pressure of the user.

16. The non-transitory computer-readable storage medium of claim 15, the instructions further comprising:
using the height difference of the wearable device as an input to the ML model.

17. The non-transitory computer-readable storage medium of claim 15,
wherein obtaining, using the first sensor data of the wearable device in the first time window, the first height of the wearable device with respect to the heart of the user comprises:
obtaining, using a camera in the first time window, a first image showing the first height of the wearable device with respect to the heart of the user;
wherein obtaining, using the second sensor data of the wearable device in the second time window, the second height of the wearable device with respect to the heart of the user comprises:
obtaining, using the camera in the second time window, a second image showing the second height of the wearable device with respect to the heart of the user; and
wherein determining the height difference of the wearable device, with respect to the heart of the user, between the first time window and the second time window comprises:
determining the height using image processing of the first image and the second image.

18. The non-transitory computer-readable storage medium of claim 15, wherein the first height and the second height of the wearable device with respect to the heart of the user are obtained from a barometer of the wearable device.

19. The non-transitory computer-readable storage medium of claim 15, wherein the blood pressure is scaled using a coefficient that indicates a change of local blood pressure due to elevating or lowering of the wearable device.

20. The non-transitory computer-readable storage medium of claim 15, wherein the blood pressure is scaled using a factor related to acceleration or deceleration.
